(19) United States
(12) Patent Application Publication
(10) Pub. No.: US 2026/0199295 A1
(43) Pub. Date: Jul. 16, 2026

(54) METHOD OF PREVENTING AND/OR TREATING METABOLIC AND/OR GASTROINTESTINAL DISORDERS

(71) Applicant: MOREHOUSE SCHOOL OF MEDICINE, Atlanta, GA (US)

(72) Inventor: Gianluca TOSINI, Atlanta, GA (US)

(21) Appl. No.: 19/445,968

(22) Filed: Jan. 12, 2026

Related U.S. Application Data
(60) Provisional application No. 63/745,081, filed on Jan. 14, 2025.

(51) Int. Cl. A61K 31/4184; A61P 3/10

(52) U.S. Cl. CPC A61K 31/4184; A61P 3/10

(57) ABSTRACT

A method of preventing and/or treating a metabolic, gastrointestinal, cardiovascular, reproductive, or psychological disorder in a subject being exposed to light-at-night, having the step of administering to the subject an effective amount of a pharmaceutical composition containing a melatonin receptor agonist having a reduced ability to cross the blood-brain barrier.

This application claims priority of U.S. Provisional Patent Application No. 63/745,081, filed Jan. 14, 2025, the entirety of which is incorporated herein by reference.

This invention was made with government support under grant number R21ES037389, awarded by the National Institutes of Health. The government has certain rights in the invention

### FIELD

The present application generally relates to medical treatment and, in particular, to treatment of disease and adverse conditions associated with light-at-night exposure.

### BACKGROUND

Melatonin (N-acetyl-5-methoxytryptamine) is a natural hormone that influences the circadian regulation of glucose and insulin levels, synchronizing the metabolism with daily feeding and fasting cycle. Genetic mutations in melatonin receptors (MT₁ and MT₂) and altered melatonin signaling have been related to increased fasting plasma glucose levels, impaired insulin secretion, and higher risk of type 2 diabetes. Melatonin administration improves liver steatosis, inflammatory markers, and lipid levels in high fat diet-induced obese (DIO) mice and ameliorates methionine- and choline-deficient diet-induced MASH in rats. Additionally, melatonin displays synergistic actions with metformin on improving adiposity and insulin sensitivity in DIO rats exhibiting circadian disruption. These findings suggest that drugs that regulate circadian disruption linked to metabolic dysfunctions could have a positive impact in the treatment of obesity, diabetes, and liver metabolic diseases.

MT₁ and MT₂ receptors mediate sleep-promotion effects and circadian rhythms (see FIG. 1, for example). It has been suggested that melatonin administration in humans could ameliorate the symptoms of insomnia, jet lag, and shift work disturbances. Moreover, based on its chronobiotic effects, melatonin has also been proposed to have a potential for ameliorating mood disorders, depression, anxiety, cancer, and Parkinson's disease. However, the therapeutic use of melatonin is limited by its poor pharmacokinetic profile.

People that work at night and are exposed to light-at-night (LAN), such as shift workers, have a higher incidence of a variety of disease states and conditions, including, but not limited to, adverse reproductive outcomes, e.g., irregular menstrual cycles, dysmenorrhea, premenstrual syndrome, and early menopause, endometriosis, infertility, miscarriage, low birth weight or pre-term delivery, and reduced breastfeeding, cardiovascular disease, including myocardial infarction, chest pain, and high blood pressure, gastrointestinal disorders, such as constipation, diarrhea, abdominal discomfort, and irritable bowel syndrome (IBS), cancer, particularly breast and colon cancer, diabetes, psychological disorders, e.g., depression, anxiety, mood swings, migraines, and stroke. In theory, melatonin could be administered to shift workers to reduce the ill effects of LAN. However, because shift workers are working at night it is impractical to administer melatonin (which promotes sleep) as a therapeutic to them. Consequently, there is a need for a therapeutic compound that can treat and/or prevent conditions associated with LAN exposure, particularly as found in shift workers.

### SUMMARY

An aspect of the present application relates to a method of preventing or treating a metabolic disorder in a subject being exposed to light-at-night, comprising the step of administering to the subject an effective amount of a pharmaceutical composition comprising the compound of Formula 1:

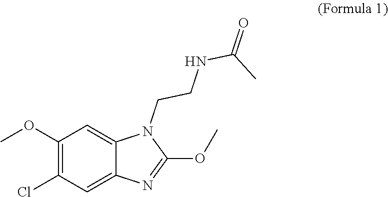 [SMILES: COc1cc2c(cc1Cl)nc(OC)n2CCNC(C)=O]

or an analog or derivative thereof.

An aspect of the present application relates to a method of preventing or treating a gastrointestinal disorder in a subject being exposed to light-at-night, comprising the step of administering to the subject an effective amount of a pharmaceutical composition comprising the compound of Formula 1:

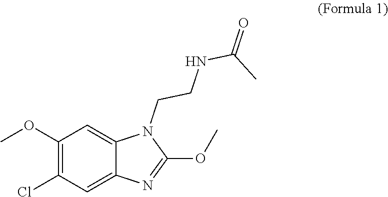 [SMILES: COc1cc2c(cc1Cl)nc(OC)n2CCNC(C)=O]

or an analog or derivative thereof.

An aspect of the present application relates to a method of preventing or treating diabetes in a subject being exposed to light-at-night, comprising the step of administering to the subject an effective amount of a pharmaceutical composition comprising the compound of Formula 1:

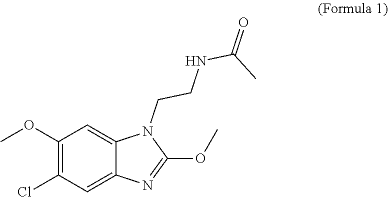 [SMILES: COc1cc2c(cc1Cl)nc(OC)n2CCNC(C)=O]

or an analog or derivative thereof.

An aspect of the present application relates to a method of preventing or treating obesity in a subject being exposed to light-at-night, comprising the step of administering to the subject an effective amount of a pharmaceutical composition comprising the compound of Formula 1:

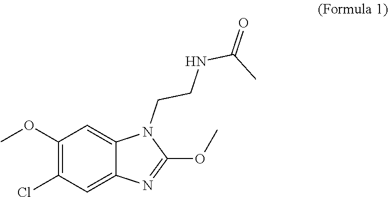 [SMILES: COc1cc2c(cc1Cl)nc(OC)n2CCNC(C)=O]

or an analog or derivative thereof.

These and other aspects, objects, features, and advantages of the example embodiments will become apparent to those having ordinary skill in the art upon consideration of the following detailed description of example embodiments.

### BRIEF DESCRIPTION OF DRAWINGS

An understanding of the features and advantages of the present invention will be obtained by reference to the following detailed description that sets forth illustrative embodiments, in which the principles of the invention may be utilized, and the accompanying drawings. The figures herein are non-limiting, for illustrative purposes only, and are not necessarily drawn to scale.

FIG. 1 shows schematic model describing the possible pathway by which melatonin via MT₁ modulates insulin sensitivity. The nocturnal increases in melatonin levels during the night activated MT₁ signaling which affect the transcription of the genes that are responsible for the assembly and the activity of P13K and thus glucose uptake.

FIGS. 2A-2F show that removal of Melatonin receptor induces changes in the regulation of Glucose metabolism and induces an increase in insulin resistance. Removal of MT₁ induces a decrease in glucose tolerance (FIG. 2A) and increase in insulin resistance (FIG. 2B) and pyruvate metabolism (FIG. 2C). FIGS. 2D, 2E, and 2F show the results of hyperinsulinemic-euglycemic clamp studies in WT and MT₁ KO mice.

FIGS. 3A and 3B show administration of melatonin during the night while the mice are exposed to LAN prevents the increase in insulin resistance that is usually observed the day after the exposure to LAN (FIG. 3A). This effect is mediated by the MT₁ KO, since administration of melatonin during the night in mice subjected to LAN prevents the increase in insulin resistance (FIG. 3B)

FIG. 4A shows that administration of Formula 1 during the night in mice exposed to LAN also prevents the increase in insulin resistance observed the day after the exposure to LAN. Administration of Formula 1 in MT₁ KO (MT₁ KO) did not provide any protection (FIG. 4B).

FIGS. 5A-5C show administration of Formula 1 does not induce change in sleep architectonic or duration. FIG. 5A shows Total Sleep, FIG. 5B shows rapid eye movement (REM) sleep, and FIG. 5C shows non-REM sleep.

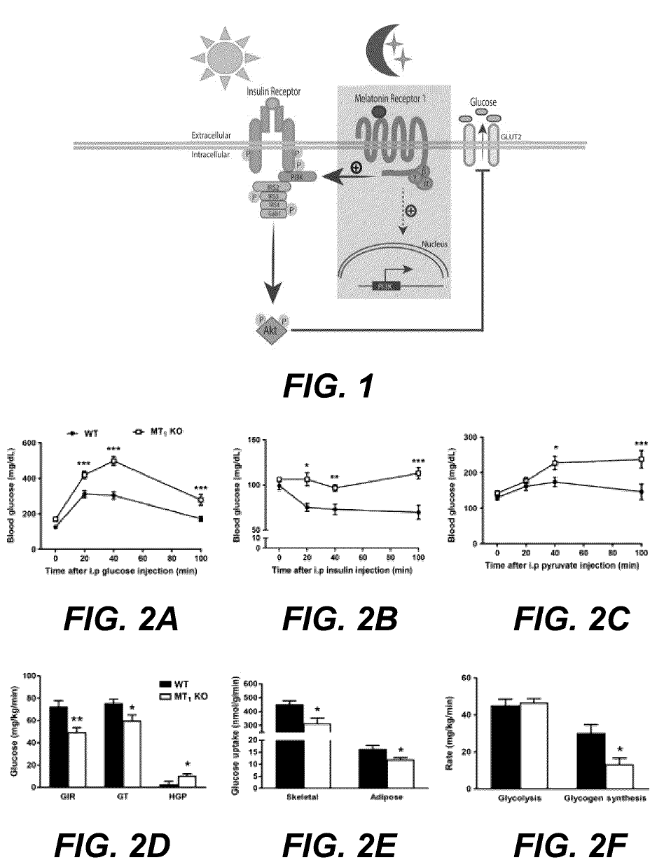

### DETAILED DESCRIPTION

The aspects of the application are described in conjunction with the exemplary embodiments, including methods, materials and examples, such description is non-limiting, and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

All publications and patents cited in this specification are cited to disclose and describe the methods and/or materials in connection with which the publications are cited. All such publications and patents are herein incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’.”

It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The recitation of numerical ranges by endpoints includes all numbers and fractions subsumed within the respective ranges, as well as the recited endpoints.

As used herein, “ZT” refers to Zeitgeber time. In chronobiology, ZT marks biological rhythms where ZT0 is “lights on” (start of the light/day cycle) and ZT12 is “lights off” (start of the dark/night cycle). ZT0-ZT12 refers to a day, or light, cycle. ZT12-ZT24 (or 0) refers to a night, or dark, cycle.

The term “benzimidazole derivative” refers to a class of heterocyclic aromatic organic compounds that contain a benzimidazole ring structure of

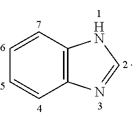

Examples of benzimidazole derivatives include, but are not limited to, compounds having the following family compound structure:

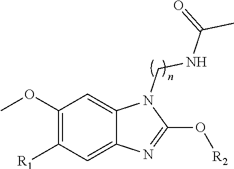

wherein R₁ may be —H, —Cl, F, Br, I, At, or Ts; wherein R₂ may be an alkyl group, and wherein n=1, 2, 3 or 4.

The term “treatment,” as used herein, refers to any type of therapy, which aims at terminating, preventing, ameliorating or reducing the susceptibility to a clinical condition as described herein. In a preferred embodiment, the term treatment relates to prophylactic treatment (i.e. a therapy to reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, or immune deficiency.

The terms “prevent,” “preventing,” or “prevention,” as used herein, refer to a method of barring a subject from acquiring a disorder and/or its attendant symptoms. In certain embodiments, the terms “prevent,” “preventing,” or “prevention” refer to a method of reducing the risk of acquiring a disorder and/or its attendant symptoms.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is a male or female.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

The term “increased level” refers to a level that is higher than a normal or control level customarily defined or used in the relevant art. For example, an increased level of immunostaining in a tissue is a level of immunostaining that would be considered higher than the level of immunostaining in a control tissue by a person of ordinary skill in the art.

An important limitation for the use of melatonin as a countermeasure for LAN resides in the fact that the administration of melatonin induces sleep and thus cannot be administered to shift workers during the night shift. Formula 1 is a benzimidazole derivative with reduced capability to cross the blood brain barrier (BBB) and—in pinealectomized rats—can rescue the change in the glucose metabolism due to the lack of melatonin signaling. However, Formula 1 can still be detected in the brain and no study has reported whether these levels have the capability to activate melatonin receptors in the brain and thus it is not known whether sleep and the entrainment of the circadian rhythms are affected by peripheral administration of Formula 1.

The present application surprisingly shows that administration of Formula 1 before the onset of light (ZT12) fully prevented changes in glucose metabolism after LAN without affecting sleep.

### Methods of the Present Application.

An aspect of the present application relates to a method of preventing and/or treating metabolic and gastrointestinal disorders in a subject exposed to LAN, comprising the step of administering to the subject an effective amount of a pharmaceutical composition comprising the compound of Formula 1 (source: ACH-000143; Aché Laboratórios Farmacêuticos, Guarulhos, São Paulo 07034-904, Brazil):

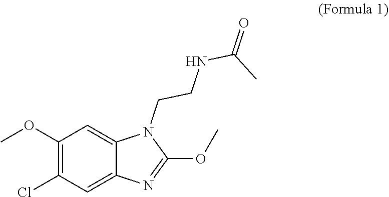 [SMILES: COc1cc2c(cc1Cl)nc(OC)n2CCNC(C)=O]

or an analog or derivative thereof.

Formula 1 (C₁₃H₁₆CIN₃O₃) is an orally active melatonin receptor agonist, with EC₅₀ values of 0.06 nM and 0.32 nM for MT₁ and MT₂, respectively. Formula 1 is a benzimidazole derivative and reduces liver triglycerides and steatosis in diet-induced obese rats and is devoid of hERG binding, genotoxicity, and behavioral alterations at doses up to 100 mg/kg p.o.

An aspect of the present application relates to a method of preventing or treating a metabolic, gastrointestinal, reproductive, cardiovascular, or psychological disorder in a subject being exposed to light-at-night, comprising the step of administering to the subject an effective amount of a pharmaceutical composition comprising a melatonin receptor agonist having a reduced ability to cross the BBB.

“Light-at-night” includes, but is not limited to, exposure to light during hours when it is typically dark because the sun has set. Sources of light-at-night may be, but are not limited to, outdoor artificial lights, such as those from houses, offices, shops, and parking lots, headlights from vehicles, displays in commercial areas, and smartphone and computer screens. Night shift workers are particularly exposed to light-at-night. Shift work generally means any arrangement of daily working hours other than standard daylight hours.

“Night shift work” or “night shift” includes, but is not limited to, working at least 3 hours between, for example, midnight and 5:00 AM. Night shift workers may work only nights (i.e., permanent night shift workers) or alternate between night, day, and evening shifts (i.e., rotating night shift workers). Forward-rotating schedules go from day to evening to night shifts, whereas backward rotating schedules go from night to evening to day shifts. Schedules can also vary in the number of consecutive days before a shift changes; fast schedules change every 2, 3, or 4 days (See, e.g., National Toxicology Program, National Institute of Environmental Health Sciences, Research Triangle Park, NC, USA. Public Health Service U.S. Department of Health and Human Services. “Running title: Night Shift Work and Light at Night and Cancer.” National Toxicology Program Cancer Hazard Assessment Report on Night Shift Work and Light at Night (April 2021; ntp.niehs.nih.gov/ntp/results/pubs/cancer_assessment/lanfinal20210400_508.pdf)).

Light-at-night exposure can lead to a condition of circadian disruption in subjects exposed to it. Light-at-night exposure is associated with the development or exacerbation in exposed subjects of a wide ranges of disorders, including, but not limited to, Metabolic and Gastrointestinal disorders, such as Insulin resistance, Metabolic Syndrome, pre-diabetes, obesity, Diabetes Mellitus (type 1 and type 2 diabetes), Glycogen Storage Disease, Hyperlipidemia, Gaucher Disease, Tay-Sachs Disease, gout, Polycystic Ovary Syndrome, non-alcoholic fatty liver disease, Pancreatitis, constipation, diarrhea, abdominal discomfort, and irritable bowel syndrome (IBS), Inflammatory Bowel Disease (IBD), Gastroesophageal Reflux Disease (GERD), Functional Dyspepsia; Cardiovascular disease, such as, myocardial infarction, high blood pressure, adverse reproductive conditions (such as, irregular menstrual cycles, dysmenorrhea, premenstrual syndrome, early menopause, endometriosis, infertility, miscarriage, low birth weight or pre-term delivery, reduced breastfeeding), psychological disorders (such as depression, bipolar disorder, hallucinations, paranoia, and disorganized thinking), and various cancers, including, but not limited to, colorectal cancer, liver cancer (such as hepatocellular carcinoma), pancreatic cancer (including insulinoma and glucagonoma), esophageal cancer, gall bladder cancer, breast cancer, uterine cancer, ovarian cancer, endometrial cancer, fibroids, and prostate cancer.

In some embodiments, the subject has one or more metabolic disorders selected from the group consisting of: Insulin resistance, Metabolic Syndrome, pre-diabetes, obesity, diabetes mellitus (type 1 and type 2 diabetes), glycogen storage disease, hyperlipidemia, Gaucher disease, Tay-Sachs disease, gout, polycystic ovary syndrome, non-alcoholic fatty liver disease, and metabolic dysfunction-associated steatohepatitis (MASH, also known as nonalcoholic steatohepatitis (NASH)).

In some embodiments, the subject has one or more gastrointestinal disorders selected from the group consisting of: pancreatitis, constipation, diarrhea, abdominal discomfort, and IBS, IBD, GERD, and functional dyspepsia.

### Route of Administration

The melatonin receptor agonist of the present application may be administered to the subject with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, the melatonin receptor agonist is administered directly to a gastrointestinal tissue, including administration directly to the tissue surface during invasive procedures.

The melatonin receptor agonist of the present application can be administered in the usually accepted pharmaceutically acceptable carriers. Acceptable carriers include, but are not limited to, saline, buffered saline, glucose in saline. Solid supports, liposomes, nanoparticles, microparticles, nanospheres or microspheres may also be used as carriers for administration of the Formula 1.

One or more of the melatonin receptor agonist of the present application discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

These therapeutic compositions may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that are apparent to those skilled in such administration. An appropriate carrier is evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

### Dosage

The appropriate dosage (“therapeutically effective amount”) of a melatonin receptor agonist of the present application of the present application will depend, for example, on the condition to be treated, the severity and course of the condition, whether the melatonin receptor agonist of the present application is administered for preventive or therapeutic purposes, previous therapy, the patient's clinical history and response to the melatonin receptor agonist of the present application, the type of melatonin receptor agonist used, and the discretion of the attending physician. The melatonin receptor agonist of the present application is suitably administered to the patent at one time or over a series of treatments and may be administered to the patent at any time from diagnosis onwards. The melatonin receptor agonist of the present application may be administered as the sole treatment or in conjunction with other drugs or therapies useful in treating the condition in question.

As a general proposition, the therapeutically effective amount of the melatonin receptor agonist of the present application, are administered in the range of about 0.01 mg/kg body weight/day to about 2500 mg/kg body weight/day whether by one or more administrations. In particular embodiments, the range of the melatonin receptor agonist of the present application administered is from about 0.1 mg/kg body weight/day to about 50 mg/kg body weight/day, from about 1 mg/kg body weight/day to about 25 mg/kg body weight/day, or from about 5 mg/kg body weight/day to about 10 mg/kg body weight/day.

In some embodiments, the daily dose of the melatonin receptor agonist of the present application is in the range of 0.01-5000 mg, 0.1-5000 mg, 1-5000 mg, 10-5000 mg, 100-5000 mg, 1000-5000 mg, 0.01-1000 mg, 0.1-1000 mg, 1-1000 mg, 10-1000 mg, 100-1000 mg, 0.01-100 mg, 0.1-100 mg, 1-100 mg, 10-100 mg, 0.01-10 mg, 0.1-10 mg, 1-10 mg, 0.01-1 mg, 0.1-1 mg, or 0.01-0.1 mg.

The melatonin receptor agonist of the present application may be administered, as appropriate or indicated, a single dose as a bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

The dosages and treatment regimens utilizing melatonin receptor agonist of the present application can be determined by the person of skill in the art. Certain of the melatonin receptor agonists of the present application are approved for use for the treatment of other conditions, and thus dosages and prescribing information is known.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations

In some embodiments, the melatonin receptor agonist of the present application is formulated for the desired route of administration using one or more pharmaceutically acceptable carriers. As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration. In certain embodiments, the pharmaceutical composition is administered directly into a target tissue.

Solutions or suspensions used for parenteral, intradermal, or subcutaneous application can include the following components: a sterile diluent such as water for injection, saline solution, fixed oils, polyethylene glycols, glycerin; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it are preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially, for example, from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved, and the limitations inherent in the art of compounding such an active compound for the treatment of individuals.

### Combination Therapy

In some embodiments, the methods of the present application further comprise the step of administering to the subject one or more additional therapeutic agents. The additional therapeutic agents may be administered prior to, concurrently with, or after, the administration of the melatonin receptor agonist of the present application.

An aspect of the present application relates to a method of preventing or treating a metabolic disorder in a subject being exposed to light-at-night, comprising the step of administering to the subject an effective amount of a pharmaceutical composition comprising the compound of Formula 1:

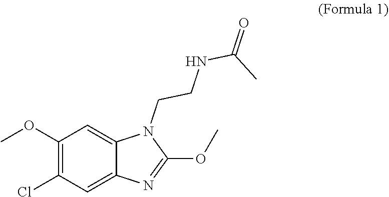 [SMILES: COc1cc2c(cc1Cl)nc(OC)n2CCNC(C)=O]

or an analog or derivative thereof.

In some embodiments, the metabolic disorder is selected from the group consisting of: insulin resistance, metabolic syndrome, pre-diabetes, obesity, diabetes mellitus (type 1 and type 2 diabetes), glycogen storage disease, hyperlipidemia, Gaucher disease, Tay-Sachs disease, gout, polycystic ovary syndrome, non-alcoholic fatty liver disease, and metabolic dysfunction-associated steatohepatitis (MASH).

In some embodiments, the pharmaceutical composition is administered orally, intravenously, intramuscularly, or subcutaneously.

In some embodiments, the pharmaceutical composition is administered at a daily dose of 0.01-2500 mg Formula 1/kg body weight. In some embodiments, the pharmaceutical composition is administered at a daily dose of 0.1-50 mg Formula 1/kg body weight.

An aspect of the present application relates to a method of preventing or treating a gastrointestinal disorder in a subject being exposed to light-at-night, comprising the step of administering to the subject an effective amount of a pharmaceutical composition comprising the compound of Formula 1:

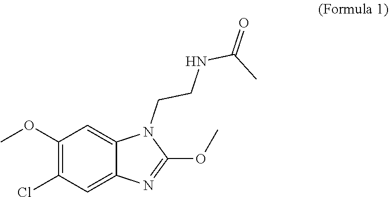 [SMILES: COc1cc2c(cc1Cl)nc(OC)n2CCNC(C)=O]

or an analog or derivative thereof.

In some embodiments, the gastrointestinal disorder is selected from the group consisting of: pancreatitis, constipation, diarrhea, abdominal discomfort, and irritable bowel syndrome (IBS), inflammatory bowel disease (IBD), gastroesophageal reflux disease (GERD), and functional dyspepsia.

In some embodiments, the pharmaceutical composition is administered orally, intravenously, intramuscularly, or subcutaneously.

In some embodiments, the pharmaceutical composition is administered at a daily dose of 0.01-2500 mg Formula 1/kg body weight. In some embodiments, the pharmaceutical composition is administered at a daily dose of 0.1-50 mg Formula 1/kg body weight.

An aspect of the present application relates to a method of preventing or treating diabetes in a subject being exposed to light-at-night, comprising the step of administering to the subject an effective amount of a pharmaceutical composition comprising the compound of Formula 1:

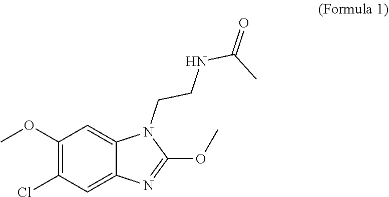 [SMILES: COc1cc2c(cc1Cl)nc(OC)n2CCNC(C)=O]

or an analog or derivative thereof.

In some embodiments, the pharmaceutical composition is administered orally, intravenously, intramuscularly, or subcutaneously.

In some embodiments, the pharmaceutical composition is administered at a daily dose of 0.01-2500 mg Formula 1/kg body weight. In some embodiments, the pharmaceutical composition is administered at a daily dose of 0.1-50 mg Formula 1/kg body weight.

An aspect of the present application relates to a method of preventing or treating obesity in a subject being exposed to light-at-night, comprising the step of administering to the subject an effective amount of a pharmaceutical composition comprising the compound of Formula 1:

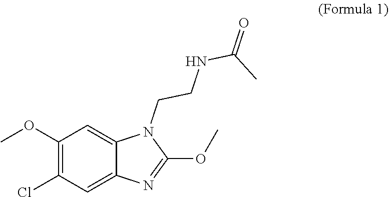 [SMILES: COc1cc2c(cc1Cl)nc(OC)n2CCNC(C)=O]

or an analog or derivative thereof.

In some embodiments, the pharmaceutical composition is administered orally, intravenously, intramuscularly, or subcutaneously.

In some embodiments, the pharmaceutical composition is administered at a daily dose of 0.01-2500 mg Formula 1/kg body weight. In some embodiments, the pharmaceutical composition is administered at a daily dose of 0.1-50 mg Formula 1/kg body weight.

An aspect of the present application relates to a method of preventing or treating cancer in a subject being exposed to light-at-night comprising the step of administering to the subject an effective amount of a pharmaceutical composition comprising the compound of Formula 1:

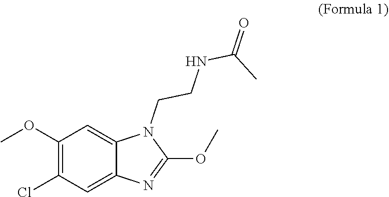 [SMILES: COc1cc2c(cc1Cl)nc(OC)n2CCNC(C)=O]

or an analog or derivative thereof.

In some embodiments, the cancer is one or more of, colorectal cancer, liver cancer (such as hepatocellular carcinoma), pancreatic cancer (including insulinoma and glucagonoma), esophageal cancer, gall bladder cancer, and breast cancer.

An aspect of the present application relates to a method of preventing or treating metabolic syndrome (MetS) in a subject being exposed to light-at-night comprising the step of administering to the subject an effective amount of a pharmaceutical composition comprising the compound of Formula 1:

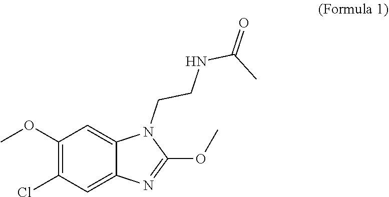 [SMILES: COc1cc2c(cc1Cl)nc(OC)n2CCNC(C)=O]

or an analog or derivative thereof.

An aspect of the present application relates to a method of preventing or treating cardiovascular disease in a subject being exposed to light-at-night comprising the step of administering to the subject an effective amount of a pharmaceutical composition comprising the compound of Formula 1:

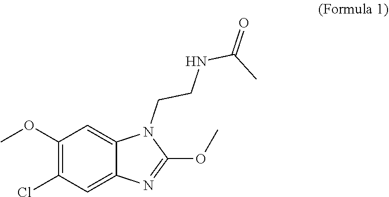 [SMILES: COc1cc2c(cc1Cl)nc(OC)n2CCNC(C)=O]

or an analog or derivative thereof.

An aspect of the present application relates to a method of preventing or treating a reproductive complication in a subject being exposed to light-at-night comprising the step of administering to the subject an effective amount of a pharmaceutical composition comprising the compound of Formula 1:

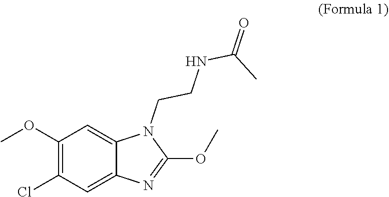 [SMILES: COc1cc2c(cc1Cl)nc(OC)n2CCNC(C)=O]

or an analog or derivative thereof.

An aspect of the present application relates to a method of preventing or treating a psychological disorder in a subject being exposed to light-at-night comprising the step of administering to the subject an effective amount of a pharmaceutical composition comprising the compound of Formula 1:

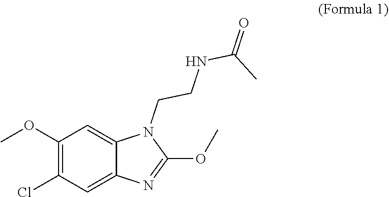 [SMILES: COc1cc2c(cc1Cl)nc(OC)n2CCNC(C)=O]

or an analog or derivative thereof.

An aspect of the present application relates to a method of preventing or treating a metabolic, gastrointestinal, cardiovascular, reproductive, or psychological disorder in a subject being exposed to light-at-night, comprising the step of administering to the subject an effective amount of a pharmaceutical composition comprising a melatonin receptor agonist having a reduced ability to cross the BBB.

The following examples are offered by way of illustration of certain embodiments of aspects of the application herein. None of the examples should be considered limiting on the scope of the application.

### EXAMPLES

### Example 1: Removal of MT₁ Signaling Induces Insulin Resistance in Mice

Removal of MT₁ induces a decrease in glucose tolerance (FIG. 2A) and increase in insulin resistance (FIG. 2B) Pyruvate metabolism (FIG. 2C). To gain a better understanding of the mechanisms underlying insulin resistance in MT₁ KO mice, this study performed hyperinsulinemic-euglycemic clamp studies in WT and MT₁ KO mice (FIGS. 2D-2F). MT₁ KO mice required a substantially lower glucose infusion rate (GIR) to maintain euglycemia during the clamp and exhibited decreased rates of glucose turnover compared to WT mice (FIG. 2D). At the tissue level, decreased insulin sensitivity in MT₁ KO mice was mostly attributed to significant reductions in insulin-stimulated glucose uptake in skeletal muscle and white adipose tissue (FIG. 2E). Additionally, pyruvate tolerance as well as insulin-mediated suppression of hepatic glucose production (HGP) during the clamp was significantly impaired in MT₁ KO mice (FIGS. 2C, 2D). Consistent with a classical insulin-resistant phenotype, MT₁ KO mice also exhibited substantial impairments in whole-body glycogen synthesis (FIG. 2F). Taken together these data point to the existence of a generalized mechanism by which the loss of MT₁ affects insulin sensitivity in multiple insulin-responsive organs.

### Example 2: Administration of Exogenous Melatonin Prevents the Decrease in Glucose Tolerance Observed After LAN

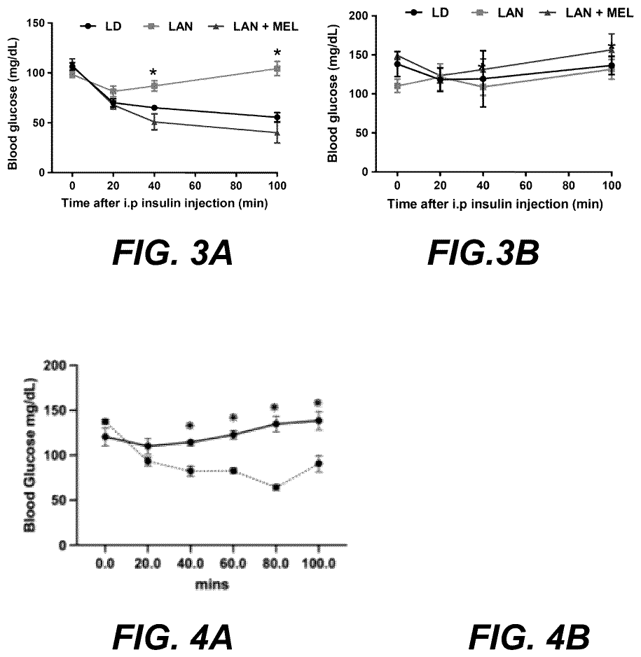

The fact that the insulin signaling is affected in MT₁ KO mice during the light period, when melatonin levels in the blood are undetectable, prompted the present investigation of whether the activation of MT₁ signaling at night is required and sufficient to modulate insulin signaling during the day. To determine whether the presence of the nocturnal peak in melatonin level at night is required to modulate insulin sensitivity during the day, WT mice were exposed to light during the night (LAN) in order to inhibit nocturnal melatonin synthesis, and then on the following day performed an IPITT at ZT6. As shown in FIG. 3A, WT mice exposed to LAN showed a significant decrease in insulin sensitivity that mimics what was observed in MT₁ KO mice (FIG. 3B). To determine whether the activation of MT₁ at night was sufficient to modulate insulin sensitivity during the day (i.e., to exclude that the exposure to light would have an effect on insulin sensitivity independently from melatonin), WT mice were exposed to LAN again, but this time, the drinking water was supplemented with melatonin (20 μg/ml) during the night (from ZT12 to ZT0) to restore the nocturnal peak in melatonin levels. As shown in FIG. 3, the supplementation with melatonin of mice exposed to constant light during the night restored insulin sensitivity, the next day at ZT6, to the same magnitude as WT mice exposed to a normal dark-light cycle. No effect of melatonin administration at night were observed in MT₁ KO (FIG. 3B).

### Example 3: Administration of the Melatonin Receptor Agonist Formula 1 Prevents the Decrease in Glucose Tolerance Observed After LAN

Formula 1 may be able to prevent the development of metabolic dysfunctions in pinealectomized rats. The present study performed an experiment to test whether administration of Formula 1 during LAN may obtain results with the administration of exogenous melatonin. Mice were injected (i.p.) with vehicle or Formula 1 (10 mg/kg) at Zeitgeber Time (ZT) 12 and exposed to LAN (300 lux fluorescent light). Then at ZT0 food was removed and animals were fasted for 6 hours, then at ZT6 an insulin tolerance test (ITT; FIG. 4A) was performed (n=6 for each vehicle and Formula 1 group; Mean±SEM; Two-way Anova, *P, 0.05). Administration of Formula 1 at ZT12 prevented the increase in insulin resistance observed in mice exposed at LAN. No significant effect of Formula 1 administration was observed in MT₁ KO mice (FIG. 4B). FIG. 4A: Circles with solid line, Formula 1 treated, Circles with dotted line mice treated with vehicle. FIG. 4B: Circles: Formula 1; squares: vehicle.

### Example 4: Administration of the Melatonin Agonist Formula 1 does not Affect Sleep Architectonic and Sleep Duration

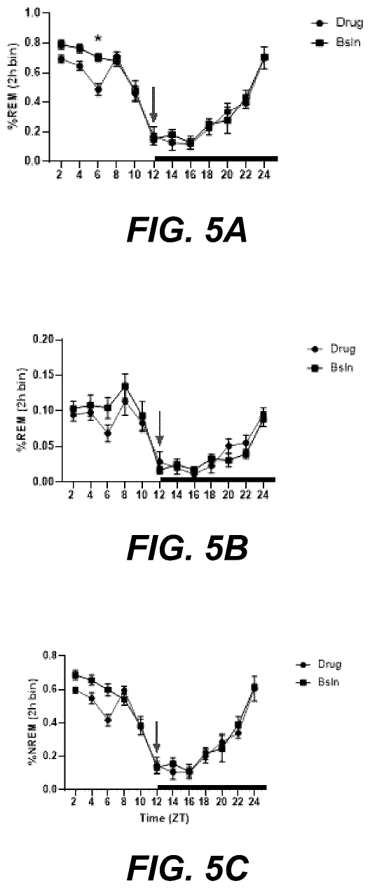

As previously mentioned, administration of melatonin promotes sleep. This study investigated if Formula 1 at the same dose (10 mg/kg) that prevents the changes in GTT and ITT after LAN (FIGS. 5A and 5B) would affect sleep. To test whether Formula 1 will alter sleep states (i.e., REM, non-REM, and Total sleep), the sleep-wake cycle after Formula 1 administration was monitored using polysomnography (PSG). Mice (n=5) in mice maintained under the 12:12 light-dark cycle. Epidural EEG electrodes were surgically implanted in anesthetized mice in the frontal cortex and visual cortex. Two EMG electrodes were placed in the neck muscle. One week after surgery, mice were moved to the sleep-recording chamber and connected to a lightweight tether attached to a low-resistance commutator mounted over the cage. This enabled complete freedom of movement throughout the cage. Except for the recording tether, conditions in the recording chamber were identical to those in the home cage. Mice were allowed a minimum of 7 additional days to acclimate to the tether and recording chamber. Mice were then injected with Formula 1 (10 mg/kg intraperitoneally) one hour before the onset of darkness. EEG/EMG data were continuously recorded for 2 days (48 hrs.). The first 24 hrs. represent the baseline recoding while the additional 24 hrs. (i.e., 12 h before and after drug injection) were used to determine the effect of Formula 1. Data acquisition was performed on a personal computer running Sirenia Acquisition software (Pinnacle Technologies), a software system designed specifically for polysomnographic recording in rodents. After collection, all waveforms will be classified by a trained observer (using both EEG leads and EMG) as wake, REM, or non-REM sleep. After scoring, analysis included standard sleep metrics including total sleep, non-REM and REM sleep time, bout length and bout duration. As shown in FIG. 5A and Table 1 (Total Sleep; post-hoc difference baseline vs. Formula 1 (Tukey's)), FIG. 5B and Table 2 (REM sleep; no significant differences with post-hoc analysis), and FIG. 5C and Table 3 (non-REM sleep; no significant differences with post-hoc analysis), administration of Formula 1 did not induce any change in the sleep architectonic or duration.

TABLE 1
Repeated Measures Analysis of Variance
(RM Anova) for Total Sleep

| Variation Source | % Total Variation | P value | Effect |
|---|---|---|---|
| Time × Formula 1 | 1.988 | 0.2727 | No |
| Time | 80.90 | <0.0001 | Yes |
| Formula 1 | 0.5808 | 0.1245 | No |

TABLE 2
RM ANOVA for REM Sleep

| Variation Source | % Total Variation | P value | Effect |
|---|---|---|---|
| Time × Formula 1 | 2.796 | 0.3981 | No |
| Time | 85.53 | <0.0001 | Yes |
| Formula 1 | 0.2369 | 0.5548 | No |

TABLE 3
RM ANOVA for Non-REM Sleep

| Variation Source | % Total Variation | P value | Effect |
|---|---|---|---|
| Time × Formula 1 | 2.073 | 0.2666 | No |
| Time | 78.94 | <0.0001 | Yes |
| Formula 1 | 0.6190 | 0.2269 | No |

### Example 5: Administration of Formula 1 Represents a Potential Treatment to Prevent the Negative Effects of LAN

As previously mentioned, melatonin acting via MT₁ affects sleep parameters (Ochoa Sanchez et al., 2011; Sharma et al., 2015; Lopez-Canul et al., 2024). As shown in Examples 3 and 4, Formula 1 can prevent the negative effects that action of melatonin in mice exposed to LAN and that Formula 1 does not affect sleep.

The present study expands the data to fully evaluate the effectiveness of Formula 1 as possible drug to prevent the negative effects of LAN on glucose metabolism. Melatonin proficient mice are single housed and acclimated to 12L:12D in an environmental chamber for one week and then are exposed to LAN for one night (see Owino et al., 2018 for details). 15 min before the exapted light transition (ZT12) mice receive an i.p. injection of melatonin (1 mg/kg), Formula 1 (10 or 30 mg/kg), or vehicle(s) only. An additional control group is represented by mice not exposed to LAN. The following day at ZT0, food is removed from the cage and at ZT6 mice are evaluated with ITT. Each of the experimental groups consists of 10 male and 10 female mice.

Does peripheral administration of Formula 1 affect sleep? To test whether Formula 1 alters sleep states (i.e., REM, non-REM, Total sleep, etc.), the study monitors the sleep-wake cycle after Formula 1 or vehicle injection using polysomnography (PSG). Mice (n=10 for each group) are maintained under the 12:12 light-dark cycle. Epidural EEG electrodes are surgically implanted in anesthetized mice in the frontal cortex and visual cortex. Two EMG electrodes are placed in the neck muscle. One week after surgery, mice are moved to the sleep-recording chamber and connected to a lightweight tether attached to a low-resistance commutator mounted over the cage. This enables complete freedom of movement throughout the cage. Except for the recording tether, conditions in the recording chamber are identical to those in the home cage. Mice are allowed a minimum of 7 additional days to acclimate to the tether and recording chamber. Mice are then injected (i.p.) with Melatonin (1 mg/Kg), Formula 1 (10 mg/kg) or Veh(s) one hour before the onset of darkness. EEG/EMG data are continuously recorded for at least 12 h before and after drug injection. Data acquisition is performed on a personal computer running Sirenia Acquisition software (Pinnacle Technologies), a software system designed specifically for polysomnographic recording in rodents. After collection, all waveforms are classified by a trained observer (using both EEG leads and EMG) as wake, non-REM or rapid eye movement (REM) sleep. After scoring, analysis includes standard sleep metrics including: non-REM and REM sleep time, bout length and bout duration. In all recordings the study also examines spectral power in the EEG waveforms in standard frequency bands (0.5-4 Hz, 5-7, 8-13 & 13-30) and the slow/theta band (2-6 Hz). In addition, the study uses heat maps of the entire frequency range to identify other bands of interest.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

What is claimed is:

1. A method of preventing or treating a metabolic disorder in a subject being exposed to light-at-night, comprising the step of administering to the subject an effective amount of a pharmaceutical composition comprising the compound of Formula 1: 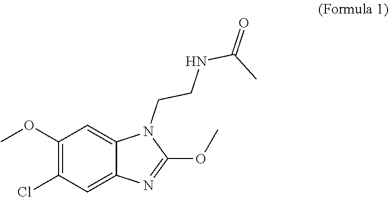 [SMILES: COc1cc2c(cc1Cl)nc(OC)n2CCNC(C)=O]
or an analog or derivative thereof.

2. The method of claim 1, wherein the metabolic disorder is selected from the group consisting of: insulin resistance, metabolic syndrome, pre-diabetes, obesity, diabetes mellitus (type 1 and type 2 diabetes), glycogen storage disease, hyperlipidemia, Gaucher disease, Tay-Sachs disease, gout, polycystic ovary syndrome, non-alcoholic fatty liver disease, and metabolic dysfunction-associated steatohepatitis (MASH).

3. The method of claim 1, wherein the pharmaceutical composition is administered orally, intravenously, intramuscularly, or subcutaneously.

4. The method of claim 1, wherein the pharmaceutical composition is administered at a daily dose of 0.01-2500 mg Formula 1/kg body weight.

5. The method of claim 1, wherein the pharmaceutical composition is administered at a daily dose of 0.1-50 mg Formula 1/kg body weight.

6. A method of preventing or treating a gastrointestinal disorder in a subject being exposed to light-at-night, comprising the step of administering to the subject an effective amount of a pharmaceutical composition comprising the compound of Formula 1: 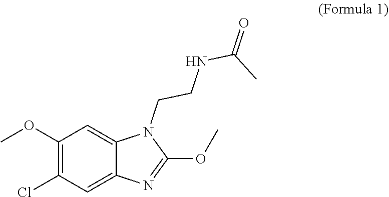 [SMILES: COc1cc2c(cc1Cl)nc(OC)n2CCNC(C)=O]
or an analog or derivative thereof.

7. The method of claim 6, wherein the gastrointestinal disorder is selected from the group consisting of: pancreatitis, constipation, diarrhea, abdominal discomfort, and irritable bowel syndrome (IBS), inflammatory bowel disease (IBD), gastroesophageal reflux disease (GERD), and functional dyspepsia.

8. The method of claim 6, wherein the pharmaceutical composition is administered orally, intravenously, intramuscularly, or subcutaneously.

9. The method of claim 6, wherein the pharmaceutical composition is administered at a daily dose of 0.01-2500 mg Formula 1/kg body weight.

10. The method of claim 6, wherein the pharmaceutical composition is administered at a daily dose of 0.1-50 mg Formula 1/kg body weight.

11. A method of preventing or treating diabetes in a subject being exposed to light-at-night, comprising the step of administering to the subject an effective amount of a pharmaceutical composition comprising the compound of Formula 1: 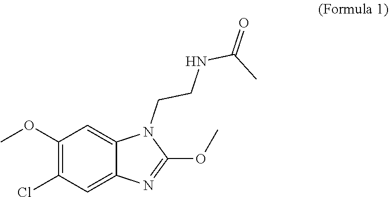 [SMILES: COc1cc2c(cc1Cl)nc(OC)n2CCNC(C)=O]
or an analog or derivative thereof.

12. The method of claim 11, wherein the pharmaceutical composition is administered orally, intravenously, intramuscularly, or subcutaneously.

13. The method of claim 11, wherein the pharmaceutical composition is administered at a daily dose of 0.01-2500 mg Formula 1/kg body weight.

14. The method of claim 11, wherein the melatonin receptor agonist is administered at a daily dose of 0.1-50 mg Formula 1/kg body weight

15. A method of preventing or treating obesity in a subject being exposed to light-at-night comprising the step of administering to the subject an effective amount of a pharmaceutical composition comprising the compound of Formula 1: 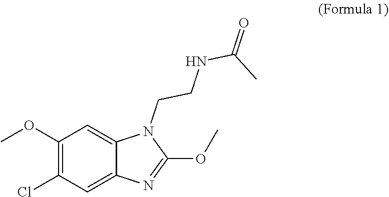 [SMILES: COc1cc2c(cc1Cl)nc(OC)n2CCNC(C)=O]
or an analog or derivative thereof.

16. The method of claim 15, wherein the pharmaceutical composition is administered orally, intravenously, intramuscularly, or subcutaneously.

17. The method of claim 15, wherein the pharmaceutical composition is administered at a daily dose of 0.01-2500 mg Formula 1/kg body weight.

18. The method of claim 15, wherein the melatonin receptor agonist is administered at a daily dose of 0.1-50 mg Formula 1/kg body weight.
